(19) United States
(12) Plant Patent Application Publication
(10) Pub. No.: US 2023/0225232 P1
(43) Pub. Date: Jul. 13, 2023

(54) Blueberry Plant Variety Named BluecsoL7

(71) Applicant: CAMPOSOL S.A., Lima (PE)

(72) Inventors: Kimberlayn Maria Apsara Sanabria Aguilar, Lima (PE); Milagros Janet Granda Mogollon de Montoya, Lima (PE); Luis José Montgomery Taboada, Lima (PE); Jorge David Romero Pajares, Lima (PE)

(73) Assignee: CAMPOSOL S.A., Lima (PE)

(21) Appl. No.: 17/803,895

(22) Filed: Jan. 12, 2023

(30) Foreign Application Priority Data
Jan. 12, 2022 (PE) 55-2022

(51) Int. Cl. A01H 6/36

(52) U.S. Cl. PLT/157

(57) ABSTRACT

A new and distinct evergreen blueberry variety with zero chill requirement named BLUECSOL7, characterized by the following combination of traits:

Strong plant vigor, semi-upright growth habit, ovate leaf shape, globose corolla shape and weak conspicuousness of ridges on corolla tube, oblata fruit shaped, extra-large fruit-size, moderate bloom intensity, medium sweetness, high acidity, and very firm fruit. Latin name of the genus and species: Vaccinium corymbosum L .

Latin Name: Vaccinium corymbosum L .

Variety denomination: Blueberry plant named BLUECSOL7

### CROSS REFERENCE TO RELATED APPLICATIONS

This application claims priority to a Peruvian Breeder’s Rights Application No. 55-2022 filed on Jan. 12, 2022, the entire contents of which is incorporated by reference herein.

### BACKGROUND OF THE INVENTION

The present invention relates to a new Vaccinium corymbosum L . plant, which was selected from open pollination between Biloxi (Vaccinium corymbosum L ., public plant) as the female (seed) parent and pollen from an unidentified variety (Vaccinium corymbosum L .). Berry seeds of plants of the Biloxi variety were collected from different fields of the CAMPOSOL S. A company, located in Viru, La Libertad, Peru at the beginnings of 2016. 11805 seedlings at high density were planted under field conditions in December of the same year.

The seedlings were evaluated during 3 growing season (2017, 2018 and 2019) and the breeding method used was phenotypic recurrent selection. In December 2019 one of the seedlings, BLUECSOL7, was isolated due to its excellent quality fruit, long postharvest life, and high yield potential. BLUECSOL7 was asexually propagated by softwood cuttings to confirm the distinctness, and stability of the characteristics observed in another growing season. 5 rooted cuttings were planted in an experimental test plot of the Blueberry Breeding Program of the CAMPOSOL S.A company, located in Viru, La Libertad, Peru. From December 2020 until the present, said test plot has shown that the unique features of this new Vaccinium variety are stable and reproduce true to type in successive generations of asexual propagation. In addition, BLUECSOL7 was subsequently propagated by softwood cutting and tissue culture and an additional plot, with 152 plants were planted in December 2021 in the same location. This new plot has maintained up to the present the distinctness, and stability characteristics of the new variety of Vaccinium corymbosum L .

### SUMMARY OF THE VARIETY

The following is summary of the description of the new and distinct variety of blueberry “BLUECSOL7” was selected in Viru, La Libertad, Peru in December 2019. BLUECSOL7 has not been observed under all environmental conditions. The phenotype may vary somewhat with variations in the environmental conditions such as temperature and light intensity, without, however, any variance in genotype. BLUECSOL7 is an evergreen blueberry variety with zero chill requirement. The following are the most distinguishing traits of this new variety: strong plant vigor, semi-upright growth habit, ovate leaf shape, globose corolla shape and weak conspicuousness of ridges on corolla tube, oblata fruit shaped, extra-large fruit-size, moderate bloom intensity, medium sweetness, high acidity, and very firm fruit.

BLUECSOL7 was selected as a mid-late variety, with high yield potential, excellent fruit quality and post-harvest life.

### BRIEF DESCRIPTION OF THE DRAWINGS

The accompanying photographs show typical bush, leaves, flower, and fruit characteristics of the new Vaccinium corymbosum L . BLUECSOL7. Colors shown are as accurate as can be reasonably reproduced by the photographic means. Photographs were taken of 2 and 4-year-old plants grown in fields of the CAMPOSOL S. A. located in Viru, La Libertad, Peru.

FIG. 1. - Shows typical bush with upright growth habit on 2-year-old plant of the new variety BLUECSOL7.

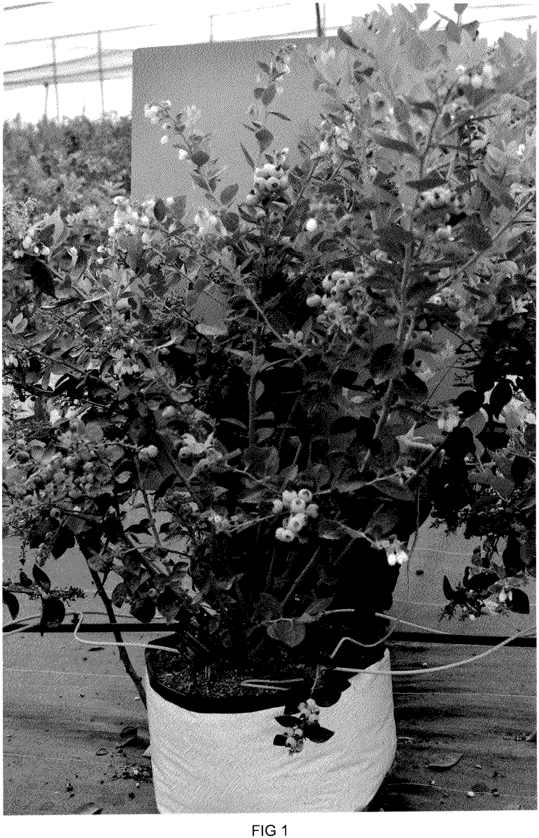

FIG. 2. - Shows the inflorescence length on 2-year-old plant of the new variety BLUECSOL7.

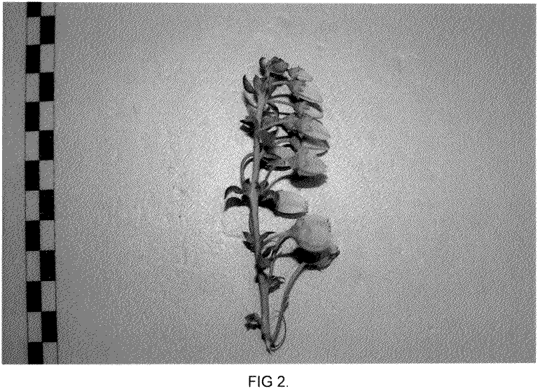

FIG. 3. - Shows the size and shape of the flower corolla on 2-year-old plant of the new variety BLUECSOL7.

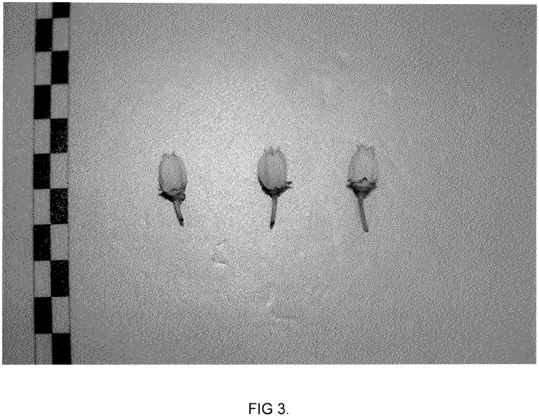

FIG. 4. - Shows the upper sides and shape of complete leaves on 2-year-old plant of the new variety BLUECSOL7.

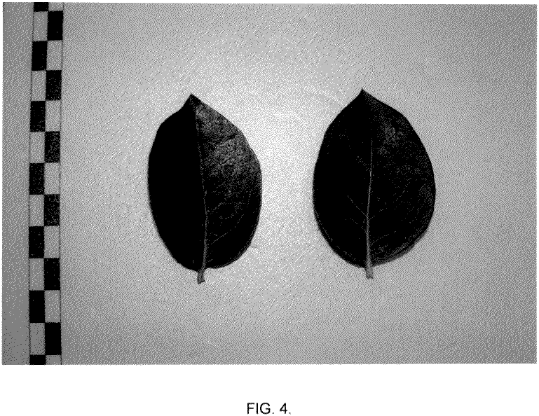

FIG. 5. - Shows the underside of a complete leave on 2-year-old plant of the new variety BLUECSOL7.

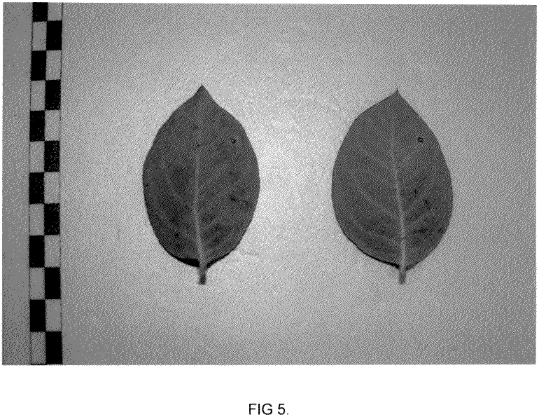

FIG. 6. - Shows the size and shape of the fruits on 2-year-old plant of the new variety BLUECSOL7.

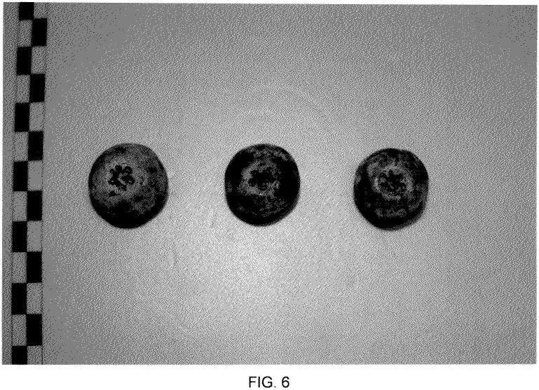

### DETAILED BOTANICAL DESCRIPTION

The following botanical description detailed forth distinctive traits of BLUECSOL7. The data were collected from clones stablished in an experimental test plot in Peru on 2-year-old plants. Color descriptions are based on the Pantone® Munsell Plant Tissue Color Book scale.

Classification:
   Family.—Ericaceae.
   Species.—Vaccinium corymbosum L .
   Common name.—Southern Highbush Blueberry.
   Variety name.—BLUECSOL7.
Parentage:
   Female parent.—Biloxi (Unpatented).
   Male parent.—Unidentified variety.
Plant:
   Vigor.—Strong.
   Growth Habit.—Semi-upright.
   Height.—Mean of 92 cm.
   Width.—Mean of 102 cm.
   Internode length.—Mean of 18.3 mm.
   Evergreenness.—Evergreen.
   Chilling requirements.—0 hours below 7° C.
   Color of 1-year.—old rough bark observed December 22.- Green Pantone® 5GY 6/6.
Propagation:
   Ease of propagation.—Propagates readily from softwood cuttings and tissue culture.
   Type.—By Tissue cultured.
   Root description.—BLUECSOL7 develops deep root systems and moderate volume of roots in the topsoil layer.
   Rooting habit.—Moderate density and branching.
Leaves:
   Length.—Mean of 56 mm.
   Width.—Mean of 33 mm.
   Length/width ratio.—1.73.
   Shape.—Ovate.
   Margin.—Serrate.
   Color on upper side of old leaves.—Green Pantone® 7.5GY ¾.
   Color on upper side of young leaves.—Green Pantone® 5GY ⅝.
Flowers:
   Flower cluster.—Medium.
   Flower fragrance.—Faint smell of geranium flowers.
   inflorescence length.—Mean of 5 cm.
   Size of corolla tube.—Mean of 11.7 mm (from pedicel attachment point to corolla tip excluding the pedicel).
   Corolla shape.—Globose.
   Style length.—Top of ovary to stigma tip. 10 mm.
Fruits:
   Diameter of calyx aperture on mature berry.—Mean of 5.7 mm.
   Diameter o depth of calyx on mature berry.—Mean of 2.6 mm.
   Detachment force for ripe berries (easy, medium, hard).—Easy.
   Berries per cluster.—About 8 to 12.
   Width.—About 18 to 24 mm.
   Weight.—Mean of 4.9 g per berry.
   Shape.—Oblate.
   Intensity of fruit bloom.—Moderate.
   Pedicel scar.—Mean of 2.5 mm.
   Color of unripe fruit.—Light green Pantone® 5GY 6/6.
   Fruit firmness.—Very firm.
   Fruit acidity.—High.
   Fruit sweetness.—Medium.
   Market use of fruit.—Fresh market.
Seeds:
   Color.—Brown Pantone® 7.5YR ⅚.
   Seeds per berry.—Mean of 7 seeds.
Reproductive organs:
   Pollen abundance.—High.
   Disease, insects, and mites.—No sensitivity to any disease and pest has been observed for BLUECSOL7.

### COMPARISON TO CLOSEST VARIETY

BLUECSOL7 is distinguished of Biloxi variety in the following characteristics:

Vigor plant in Biloxi (Public patent) is medium and in BLUECSOL7 is strong.

Ratio length/width of the leaf in BLUECSOL7 is higher than Biloxi (Public patent).

Anthocyanin coloration in the flower bud in BLUECSOL7 is weaker than Biloxi (Public patent).

Fruit size of Biloxi (Public patent)) is medium, whereas in BLUECSOL7 fruit size large fruits.

Depth of calyx basin on mature berry is deep in Biloxi (Public patent) and medium in BLUECSOL7.

What is claimed is:

1. A new and distinct blueberry plant named BLUECSOL7 substantially as illustrated and described.
